(19) United States
(12) Patent Application Publication
(10) Pub. No.: US 2003/0149011 A1
(43) Pub. Date: Aug. 7, 2003

(54) Methods and reagents for extracorporeal immunomodulatory therapy

(76) Inventors: Samuel Ackerman, Weston, MA (US); Clark M. Edson, Watertown, MA (US); Loren Fast, Foster, RI (US); Andrei Purmal, Waltham, MA (US)

Correspondence Address: MINTZ, LEVIN,, COHN, FERRIS, GLOVSKY and POPEO, P.C., One Financial Center, Boston, MA, 02111, US

(21) Appl. No.: 10/036,768

(22) Filed: Nov. 6, 2001

Related U.S. Application Data
(60) Provisional application No. 60/246,201, filed on Nov. 6, 2000.
(60) Provisional application No. 60/329,462, filed on Oct. 15, 2001.

(51) Int. Cl. A61K 31/396

(52) U.S. Cl. 514/183; 424/93.7

(57) ABSTRACT

The invention features methods for altering the immunomodulatory activity of compositions containing peripheral blood mononuclear cells. The methods include the steps of contacting PBMCs with an amount of an aziridino-containing compound effective to react with and modify the PBMC nucleic acid.

### RELATED APPLICATIONS

This application claims priority to U.S. Ser. No. 60/______, filed Oct. 15, 2001, and U.S. Ser. No. 60/246,201, filed Nov. 6, 2001. The contents of these applications are incorporated herein by reference in their entireties.

### BACKGROUND OF THE INVENTION

The invention relates to the field of immunomodulation.

Extracorporeal photopheresis is a leukapheresis-based immunomodulatory therapy that is used to treat advanced cases of several T-cell mediated diseases. In one example, peripheral blood mononuclear cells collected by apheresis are exposed to a photosensitizing compound (e.g. 8-methoxypsoralen) and UVA (ultraviolet light of wavelength between 320 and 400 nm), which together trigger a series of changes in the cells, including induction of T-cell apoptosis and macrophage cell activation. The consequence of these changes on recipient immune responses when the treated cells are reinfused into the recipient depends on the patient's disease. Photopheresis-induced immunomodulation has been proposed as a treatment of cutaneous T-cell lymphoma (CTCL), graft versus host disease (GVHD), allograft rejection following organ transplantation, and autoimmune disease (e.g. systemic lupus erythematosus, systemic sclerosis, rheumatoid arthritis).

Transfusion-induced immunomodulation can result in the production of alloantibodies, an increased infection rate, increase in the incidence of tumor relapse at least for some types of tumors, an enhanced survival of transplanted organs, and, occasionally, transfusion-associated graft-versus-host disease. The number of peripheral blood mononuclear cells (PBMCs) present in a blood product has been identified as an important contributing factor to transfusion-induced immunomodulation. Thus, approaches to deplete PBMCs using filters (leukoreduction) or to render the PBMCs functionally incompetent have been developed (e.g., treatment with UVB light (ultraviolet light between 280 and 320 mm or psoralens and UVA light), and these approaches have been found to successfully modify immunomodulatory events following transfusion.

### SUMMARY OF THE INVENTION

We have discovered that ethyleneimine oligomer treatment renders PBMCs unable to proliferate in response to phytohemagglutinin (PHA) or allogeneic stimulator cells, or to serve as stimulator cells in a mixed lymphocyte culture (MLC) reaction. These findings indicate that aziridino-containing compounds (e.g., ethyleneimine dimer, trimer, tetramer and other oligomers) are useful for extracorporeal immunomodulatory therapy for the treatment or prevention of T-cell-mediated diseases.

Accordingly, the invention features a method for treating a patient having an immune dysfunction, including the steps of treating blood compositions. An exemplary blood composition is a composition that includes PBMCs. The PBMC is contacted with an amount of an aziridino-containing compound effective to react with and modify the PBMC nucleic acid, and then administering the PBMCs to the patient. Also featured is a method for preventing immune dysfunction in a subject, including the steps of treating PBMCs with an amount of an aziridino-containing compound effective to react with and modify the PBMC nucleic acid, and then administering the PBMCs to the patient. The PBMCs may be autologous (i.e., obtained from the patient being treated) or heterologous (i.e., obtained from a donor who may be syngeneic or allogeneic with the patient). Also provided by the invention are blood compositions produced by the herein described methods.

In a related aspect, the invention features a method for reducing the immunomodulatory activity of a blood product containing PBMCs, including the steps of treating the blood product with an amount of an aziridino-containing compound effective to react with and modify the PBMC nucleic acid, and then administering the PBMCs to the patient.

It is desirable to separate the PBMCs or blood product from the aziridino-containing compound alter treatment and before administration to the patient. Methods for separating aziridino-containing compounds from biological compositions such as blood, plasma, or other PBMC-containing compositions may include for example, cell washing procedures, filtration, dialysis, affinity chromatography, ionic exchange, and the like. Some of these methods are described in copending U.S. Ser. Nos. 09/161,078, 09/260,375,09/237,136, 60/263,407 and 09/827,491 cach of which is hereby incorporated by reference. Preferably, at least 99% of the unreacted aziridino-containing compound is separated from the leukocytes to be administered to the patient.

Aziridino-containing compounds useful in the methods of the invention preferably contain a moiety having the formula (I):

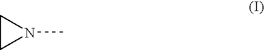

In this three-membered ring, the two carbons are preferably unsubstituted (i.e., they contain hydrogens), but they can be substituted with aliphatic or aromatic hydrocarbon moieties, each containing between one and four carbon atoms, inclusive.

In one set of examples, the aziridino-containing compound has the formula (II):

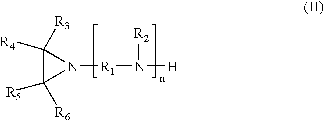

wherein each R₁ is a divalent hydrocarbon moiety containing between two and four carbon atoms, inclusive; each of R₂, R₃, R₄, R₅, and R₆ is, independently, H or a monovalent hydrocarbon moiety containing between one and four carbon atoms, inclusive; and it is an integer between one and ten, inclusive.

In various preferred embodiments, each R₁ contains two or three carbon atoms; each of R₂, R₃, R₄, R₅, and R₆ is H; and n is one or two. For example, ethyleneimine tetramer fits formula (II) when R₁ contains four carbon atoms, and each of R₂, R₃, R₄, R₅, and R6 is H, and n is one. Similarly, ethylene trimer fits formula (II) where R₁ contains three carbon atoms, each of R₂, R₃, R₄, R₅, and R₆ is H, and n is one, and ethylene dimer fits formula (II) when R₁ contains two carbon atoms, and each of R₂, R₃, R₄, R₅, and R₆ is H, and n is one.

In another set of examples, the compound has the formula (III):

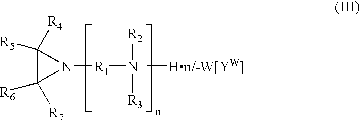

wherein each R₁ is a divalent hydrocarbon moiety containing between two and four carbon atoms, inclusive; each of R₂, R₃, R₄, R₅, and R₆, and R₇ is, independently, H or a monovalent hydrocarbon moiety containing between one and four carbon atoms, inclusive; Y is pharmaceutically acceptable counter anion; W is the valency of Y; and n is an integer between one and ten, inclusive.

Aziridino-containing compounds also include open-ring counterparts to the compounds of formula (I). In one example, aziridino-containing compounds useful in the methods of the invention have the formula (IV):

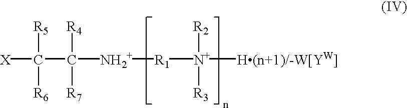

wherein each R₁ is a divalent hydrocarbon moiety containing between two and four carbon atoms, inclusive; each of R₂, R₃, R₄, It₅, R₆, and R₇ is, independently, H or a monovalent hydrocarbon moiety containing between one and four carbon atoms, inclusive; X is Cl or Br; Y is a pharmaceutically acceptable counter anion; W is the valency of Y; and n is an integer between one and ten, inclusive.

In various preferred embodiments of compounds satisfying formula (III) or formula (IV), each R₁ contains two or three carbon atoms; each of R₂, R₃, R₄, R₅, and R₆ is H; and n is one or two. Suitable counter anions include nitrate, sulfate, halide, phosphate, and tosylate ions.

In another set of examples, the aziridino-containing compound has one of the following chemical structures.

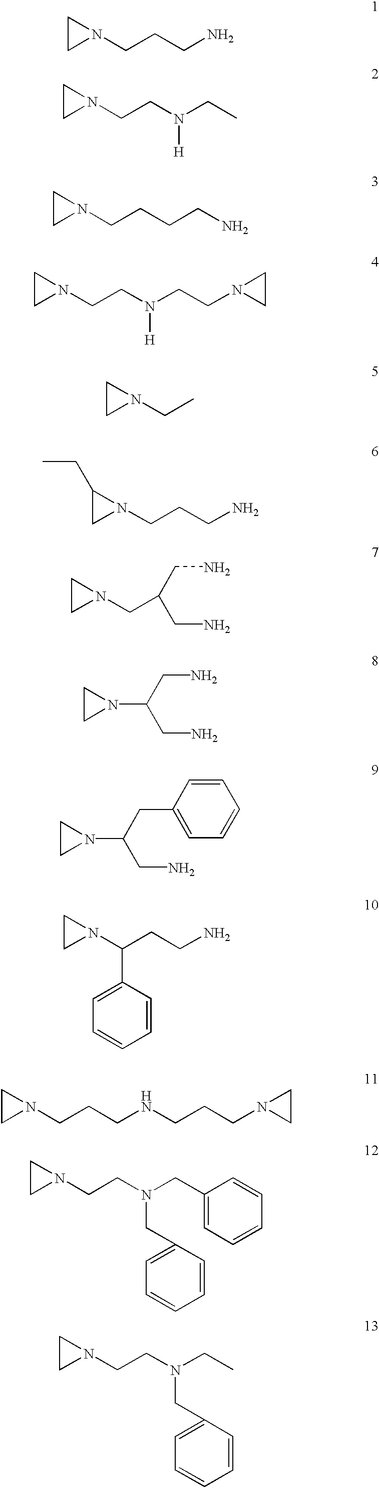

By “valency” is meant the positive or negative integer that represents the combining capacity of an atom or a radical (determined by the number of electrons that it will lose, add, or share when it reacts with other atoms). For example, the valency of a chloride anion is −1, while the valency of a sulfate anion is −2.

In certain embodiments, the ethyleneimine oligomer inactivating agent is a trimer, linear tetramer or branched tetramer.

Immune dysfunctions that can be treated and/or prevented using the methods of the invention include, for example, cutaneous T-cell lymphoma, graft versus host disease including transfusion associated graft versus host disease, allograft rejection following transplantation, systemic lupus erythematosus, systemic sclerosis, and rheumatoid arthritis. In a particularly preferred embodiment, the methods of the invention inhibit induction of chronic or acute graft versus host disease in a recipient.

In a preferred embodiment, the invention includes a method for preventing or treating graft-versus-host (GVH) disease in a patient by (a) extracorporeally treating a blood composition with an effective amount of any of herein described aziridino-containing compound; and (b) administering the treated blood cell population to the patient, thereby preventing GVH disease in the patient.

If desired, the method may further include removing the aziridino-containing compound from the heterologous treated blood composition. Preferably, at least 99% of the aziridino-containing compound is removed from the treated blood cell composition prior to administering the treated blood composition to the patient.

The blood composition can include, e.g., peripheral blood mononuclear cells (PBMC). Alternatively, or in addition, the blood composition can include a non-leukoreduced blood cell concentrate. Alternatively, or in addition, the blood composition can be a heterologous blood cell population. The patient is preferably a human. In some embodiments, the patient suffers from or is at risk for immune dysfunction.

Also provided by the invention is a method for treating graft-versus-host (GVH) disease in a patient by: (a) treating a heterologous blood composition with an effective amount of an aziridino-containing compound; and (b) administering the treated blood cell population to the patient, thereby treating GVH disease in the patient.

In another aspect the invention includes a method of treating an alloantibody response in a patient by (a) treating a heterologous blood composition with an effective amount of an aziridino-containing compound (such as the aziridino-containing compounds disclosed herein, including ethylene oligomers); and (b) administering the treated blood cell population to the patient, thereby preventing the alloantibody response in the patient.

In a further aspect the invention provides a method for functionally inactivating a leukocyte in a patient. The method includes treating a heterologous blood composition comprising a leukocyte with an effective amount of an aziridino-containing compound (such as the aziridino-containing compounds disclosed herein, including ethylene oligomers); and administering the treated blood cell population to the patient, thereby inactivating the leukocyte in the patient. Preferably, the leukocyte does not proliferate following treatment with the aziridino-containing compound.

Also provided by the invention are blood compositions produced by the herein described methods, as well as a pharmaceutical composition for treating immune dysfunction in a patient, the pharmaceutical formulation comprising a therapeutically effective, non-toxic amount of an aziridino-containing compound and a pharmaceutically acceptable carrier. In some embodiments, the aziridino-containing compound is an ethylene oligomer, e.g., an ethyleneimine dimer, ethyleneimine trimer, or ethylene tetramer.

In preferred embodiments of the invention, production of alloantibodies and/or the generation of restricted CTL is reduced or substantially inhibited by using methods of the invention which include a step of treating blood compositions comprising PBMCS, such as red blood cell concentrates, with azirdino comprising compounds of the invention. In a preferred embodiment, cells comprising nucleic acids, such as PBMCs, spleen cells, treated with the aziridino-comprising compounds of the invention are functionally inactivated.

One advantage of using aziridino-containing compounds according to the invention is that the treatment of blood components does not require the use of UV light banks.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

We have shown that ethyleneimine oligomer, while not immediately toxic to PBMCs in treated blood, renders PBMCs unable to proliferate in response to stimuli or to stimulate immune responses. Cells underwent apoptosis within 24 hours following treatment. Other aziridino-containing compounds (or their open chain counterparts) can be used, as described above, to similarly affect PBMCs.

The ability of aziridino-containing compounds to functionally inactivate PBMCs is similar to that reported for psoralen combined with UVA light exposure, which induces apoptosis in the exposed cells. Thus, treating blood components with aziridino-containing compounds can modify recipient immune responses in a similar fashion to what has been reported when using psoralen in combination with UVA treated components.

The method can additionally be used to eliminate leukocytes from any blood-derived composition. As used herein, the terms “blood-derived compositions” and “blood compositions” are used interchangeably and are meant to include whole blood, blood fractions, e.g. blood plasma, blood plasma fractions, blood plasma precipitate (e.g., cryoprecipitate, ethanol precipitate or polyethylene glycol precipitate), blood plasma supernatant (e.g., cryosupernatant, ethanol supernatant or polyethylene glycol supernatant), solvent/detergent (SD) plasma, platelets, intravenous immunoglobulin (IVIG), IgM, purified coagulation factor concentrate, fibrinogen concentrate, or various other compositions which are derived from human or animal. Blood-derived compositions also include purified coagulation factor concentrates (e.g., factor VIII concentrate, factor IX concentrate, fibrinogen concentrate, and the like) prepared by any of various methods common in the art including ion exchange, affinity, gel permeation, and/or hydrophobic chromatography or by differential precipitation. In some embodiments, the aziridino-containing compounds are used to treat whole blood, blood fractions, e.g. platelet concentrates or red blood cell-enriched blood fractions, including red blood cell concentrates.

Unless indicated otherwise, an “effective amount” as used herein is an amount sufficient to inhibit replication of nucleic acid in a nucleated blood cell, e.g., a leukocyte. In some embodiments, an effective amount is a solution that includes 0.001% to 0.1% of the aziridino-containing compound. For example, the aziridino-containing compound may be present at a concentration of 0.01% to 0.1% or 0.05% to 0.1%.

In some embodiments, an aziridino-containing compound is contacted with a blood composition for 60 to 1440 minutes of the aziridino-containing compound. For example, the aziridino-containing compound may be present at a period of 1200 minutes to 1440 minutes or 1260 to 1380 minutes. In some embodiments, a non-viricidal amount of an aziridino-containing compound is used, i.e., the amount of aziridino-containing compound used is substantially less than that required to inactivate viral pathogens.

As used herein, a “patient” refers to any warm-blooded animal, preferably a human, non-human primate, a dog, cat, horse, cow, or sheep. A patient may be afflicted with disease, or may be free of detectable disease. Accordingly, the methods disclosed herein may be used prophylactically and/or therapeutically to treat immune dysfunction.

Isolating Peripheral Blood Mononuclear Cells

PBMCs can be prepared using methods known in the art. For example, PBMCs can be prepared from whole blood samples by separating mononuclear cells from red blood cells. There are a number of methods for isolating PBMC, e.g., velocity sedimentation, isopyknic sedimentation, affinity purification, and flow cytometry. Typically, PBMC are separated from red blood cells by density gradient (isopyknic) centrifugation, in which the cells sediment to an equilibrium position in the solution equivalent to their own density. For density gradient centrifugation, physiological media should be used, the density of the solution should be high, and the media should exert little osmotic pressure. Density gradient centrifugation uses solutions such as sodium ditrizoate-polysucrose, Ficoll, dextran, and Percoll (see, e.g., Freshney, Culture of Animal Cells (3rd ed. 1994)). Such solutions are commercially available, e.g., HISTOPAQUE® (Sigma).

For example, anticoagulated whole blood or plasma can be layered onto the gradient and centrifuged according to standard procedures (see, e.g., Fish et al., J. Virol. 69:3737-3743 (1995)). Using, e.g., the procedure in Fish et al., the red blood cells and granulocytes form a pellet, while lymphocytes and other mononuclear cells such as monocytes remain at the plasma/density gradient interface (see, e.g., Freshney, Culture of Animal Cells (3rd ed. 1994)).

A preferred method for isolating PBMC is using an apheresis device. An example of an apheresis device is a CS3000 (Baxter Fenwal).

Immune Dysfunction

The compounds described herein are useful for the treatment and/or prevention of immune dysfunction that results from the introduction of heterologous cells or tissue (e.g., following transplantation of bone marrow stem cells or solid organs such as a heart, transfusion of blood products), and that accompanies diseases such as cutaneous T-cell lymphoma and a variety of autoimmune diseases. For illustrative purposes, a few examples are provided below.

Graft-versus-host Disease

Graft-versus-host disease (GVHD) is a major complication affecting overall prognosis after allogeneic stem cell transplantation. GVHD may be classified as acute or chronic. In acute GVHD, the principal targets include the immune system, skin, liver, and intestine. Chronic GVHD is a multi-organ autoimmune-like syndrome affecting mainly the skin and the mucous membranes. In addition to the foregoing clinical manifestations, the development of acute and chronic GVHD and the immunosuppression used for GVHD prophylaxis represent significant risk factors for bacterial, viral, and fungal infections.

According to one embodiment of the invention, GVHD is treated by extracorporeally treating the patient's blood with an ethyleneimine oligomer or other compound of the invention. In this embodiment, the patient's blood is removed and separated into leukocyte-depleted blood, which is returned to the patient and leukocyte-enriched plasma, which is exposed to an effective concentration of the compound of the invention (e.g., ethyleneimine oligomer). The aziridino moiety reacts with the nucleic acid in the PBMCs, resulting in the eventual death of the cells. After the leukocyte-enriched plasma is separated from the unreacted compound (e.g., by a standard method employing a solid support), it is reinfused into the patient. The cells will die over a period of six to 24 hours, and so are administered during that interval when they can stimulate an anti-idiotypic T suppressor response, which reduces the concentrations of cytokines such as TNF-alpha and IL-2 or induce the production of antiinflammatory cytokines that inhibit the GVHD responses.

Immunosuppression after Organ Transplantation

Inhibition of allograft rejection according to the invention, which is directed at suppressing donor-specific T-cell clones in the recipient to reduce graft rejection, may reduce undesirable side effects of present methods. This method is performed in a manner similar to that described above for inhibition of graft-versus-host disease.

Other Diseases

The following diseases and conditions are also likely treatable with the extracorporeal treatment of blood cells with ethyleneimine oligomers: inflammatory bowel disease, systemic lupus erythematosus; rheumatoid arthritis; system sclerosis; and cutaneous T-cell lymphoma.

Transfusion-induced Immunomodulation

Treatment of the nucleated PBMCs present in blood components with aziridino-containing compounds results in the elimination of the cells that are responsible for transfusion induced immunomodulation that is observed following the transfusion of blood products. Accordingly, the method of the invention can be used to reduce the immunomodulatory activity of blood products, such as those described in U.S. Pat. No. 6,136,586, hereby incorporated by reference. While not wishing to be bound by theory, it is believed that aziridino-containing compounds induce DNA breakage and/or apoptosis in the treated PBMCs.

Accordingly, in a preferred embodiment of the invention, transfusion associated graft versus host disease is treated and/or prevented by treating a donor, non-leukoreduced, red blood cell concentrate with an effective amount of an ethyleneimine oligomer, e.g. dimer, trimer or tetramer under appropriate conditions which include an appropriate incubation period. An appropriate incubation period for example, may comprise a period sufficient to functionally inactivate PBMCS, e.g. a period of time sufficient to inhibit a proliferative response. The ethyleneimine oligomer is optionally separated from the red blood cell concentrate following treatment by methods which may include for example: cell washing procedures, standard procedures employing a solid support, etc. The treated red blood cell concentrate is then infused to a recipient/patient.

Treatment

In some embodiments, e.g., where autologous PBMCs are used, the combined method of leukapheretic processing and treatment using aziridino-containing compounds is preferably performed once or twice per week every one to four weeks. In certain instances, the method is performed for a few months, but preferably, the treatment is continued for four to forty months. While the particular treatment regimen will vary based on the health of the patient, it is preferred that 100-500 ml of leukocytes are treated for each day of treatment. Each individual treatment is expected to last three to four hours. In order to optimize results, it may be preferable to perform the treatment on two consecutive days.

The invention will be further illustrated in the following non-limiting examples.

### EXAMPLE 1

### Ethyleneimine Oligomer has no Effect on the Subsets of Peripheral Blood Mononuclear Cells

We first determined whether ethyleneimine oligomer has an effect on PBMCs. Ethyleneimine oligomer was added in varying concentrations (0, about 0.025%, about 0.05%, about 0.075%, or about 0.1%) to whole human blood and incubated for 0, 1, 2, or 6 hours at room temperature. Following incubation, the number and composition of PBMCs was determined. Incubation of blood with the highest concentration tested of ethyleneimine oligomer for the longest period of time tested did not affect cell count; however, cell proliferation was inhibited. Table 1 depicts data from cells incubated for 6 hours in the absence or presence of 0.1% ethyleneimine oligomer. Similar results were obtained from all other doses and timepoints.

TABLE 1

| | no ethyleneimine | 0.1% ethyleneimine |
|---|---|---|
| | oligomer | oligomer |
| PBMCs (thousands/mm³) | 7.0 | 7.6 |
| PMN (%) | 78 | 72 |
| Lymphocytes (%) | 16 | 20 |
| Monocytes (%) | 4 | 4 |
| Eosinophils (%) | 2 | 2 |
| Basophiles (%) | 0 | 0 |
| Bands (%) | 0 | 2 |
| PHA proliferation (cpm) | 110888 ± 14454 | 242 ± 48 |

### EXAMPLE 2

### Inhibition of PHA-induced Proliferation by Ethyleneimine Oligomer

While not wishing to be bound by theory, we hypothesized that, although it was not immediately toxic for the PBMCs in the treated blood, ethyleneimine oligomer could still be interfering with the proliferative ability of the PBMC because of its ability to irreversibly bind to nucleic acids. We thus tested PBMCs for their ability to proliferate in response to an optimal dose of PHA, as measured by thymidine incorporation on day 3, following treatment with ethyleneimine oligomer. Incubation of blood with about 0.1% ethyleneimine oligomer for 6 hours completely inhibited the ability of PBMCs isolated from the treated blood samples to proliferate in response to PHA (Table 1). Additional experiments demonstrated that even a one hour incubation with about 0.1% ethyleneimine oligomer was sufficient to completely inhibit the proliferative response.

To further quantitate the proliferation-inhibiting effects of ethyleneimine oligomer, the response of the treated cells to differing concentrations of ethyleneimine oligomer was tested. Incubation of blood with a log dose curve of ethyleneimine oligomer on PHA-induced proliferation was measured. The ethyleneimine oligomer dose sufficient to inhibit proliferation was between about 0.009% and about 0.09%. Dose curves using smaller increments found that there was a dose response curve in which about 0.01% ethyleneimine oligomer was sufficient to achieve complete inhibition while a concentration of 0.075% ethyleneimine oligomer gave half-maximal inhibition.

### EXAMPLE 3

### Ethyleneimine-treated PBMCs are Incapable of Functioning as Responder or Stimulator Cells

PBMCs have the ability to respond strongly to allogeneic MHC molecules. This response can be measured in vitro by mixing PBMCs from two different individuals. Because the combination would result in a two-way reaction, the assay is modified by treating one set of cells with a treatment that prevents cell proliferation of that population. These treated cells are referred to as the stimulator cells, while the untreated cells are the responder cells.

The effect of incubation with ethyleneimine oligomer on the ability of the treated PBMCs to serve as responder or stimulator cells in a MLC was also tested. PBMCs were isolated from donor blood incubated with or without ethyleneimine oligomer (0.1%, three hours) and then used as responders in a MLC. Stimulator cells were either PBMCs incubated with ethyleneimine oligomer or control PBMCs which had been treated with mitomycin C. Stimulator cells were either syngeneic to the responder cells or were allogeneic ceils. Proliferation was measured on day 4.

TABLE 2

| | Stimulator | Stimulator | |
|---|---|---|---|
| Responder Treatment | Disparity | Treatment | Proliferation |
| Untreated | syngeneic | Mitomycin C | 1726 ± 778 |
| Ethyleneimine | syngeneic | Mitomycin C | 531 ± 145 |
| oligomer | | | |
| Untreated | syngeneic | Ethyleneimine | 443 ± 23 |
| | | oligomer | |
| Untreated | allogeneic | Mitomycin C | 30053 ± 9796 |
| Ethyleneimine | allogeneic | Mitomycin C | 527 ± 100 |
| oligomer | | | |
| Untreated | allogeneic | Ethyleneimine | 504 ± 214 |
| | | oligomer | |

Ethyleneimine oligomer treatment was found to totally abrogate the response of PBMC to allogeneic stimulator cells (Table 2). Because ethyleneimine oligomer treated cells remained viable but did not exhibit any proliferation, they could potentially have served as stimulator cells. When ethyleneimine oligomer treated cells were used as stimulator cells in the MLC, however, they were found to be unable to stimulate the responder cells to proliferate (Table 2).

Our results indicate that ethyleneimine oligomer-treated cells were unable to proliferate in response to stimuli such as PHA or allogeneic stimulator cells. To quantitate the degree of inhibition, a limiting dilution analysis (LDA) was performed using untreated or ethyleneimine oligomer-treated responder cells. In this assay the number of clones that were present on day 21 reflects the residual number of cells that were still viable following treatment with ethyleneimine oligomer. The results of these assays (Table 3) indicated that no clones could be detected even when 100,000 ethyleneimine oligomer-treated cells were added per well in the responder assay, in contrast to the high frequency of clones observed in the control assays.

TABLE 3

| | | Frequency |
|---|---|---|
| | | (ethyleneimine |
| Replicate | Frequency (control) | oligomer treated) |
| 1 | 1/74 | NCD at 10⁵ |
| 2 | 1/105 | NCD at 10⁵ |
| 3 | 1/53 | NCD at 10⁵ |

### EXAMPLE 4

### Ethyleneimine-mediated PBMC Apoptosis

While not wishing to be bound by theory, we postulated that the reason ethyleneimine oligomer treated cells could not act as stimulator cells and did not proliferate was that the cells were undergoing apoptosis following incubation with ethyleneimine oligomer. One of the first events that occurs following induction of apoptosis is the appearance of phosphotidylserine on the cell surface. The presence of phosphotidylserine can be detected by annexin V staining; as cells progress through apoptosis they become permeable and stain with propidium iodide. PBMCs treated with growth medium, mitomycin C, or ethyleneimine oligomer (0.1%) were cultured in vitro for 20 hours and then stained using FITC labeled annexin V and propidium iodide. The results indicate that the ethyleneimine oligomer-treated cells exhibited much higher numbers of apoptotic cells at 20 hours of in vitro incubation than mitomycin C treated cells. To confirm the induction of apoptosis, a TUNEL staining assay (which detects fragmented DNA) was also performed on cells treated with ethyleneimine oligomer or mitomycin C. We observed greatly increased TUNEL staining in ethyleneimine oligomer-treated cells after 20 hours incubation, relative to mitomycin C treated cells, confirming the induction of apoptosis in the ethyleneimine oligomer-treated cells.

The foregoing results were achieved using the following materials and methods.

Media

RPMI 1640 medium was used as the base growth medium for all experiments. For PHA blastogenesis and mixed lymphocyte cultures (MLC), this medium was supplemented with 20% heat-inactivated fetal calf serum (FCS), L-glutamine (2 mM), and 100 units penicillin/mL and 100 μg streptomycin/mL, and referred to as R20 medium. R20 medium was made 20% v/v with dimethylsulfoxide (DMSO) for use in freezing down the γ-irradiated allogeneic pool of cells to be used in the limiting dilution assay (LDA). Two other media were used for LDA. First, they γ-irradiated allogeneic pool of PBMC were thawed and resuspended in 2×T-cell medium consisting of 80% RPMI 1640, 20% FCS, 20% human T-cell growth factor (Hu TCGF; Cellular Products Inc. Buffalo, N.Y.), 100 units/mL of recombinant human interleukin-2 (rhuIL-2, Cellular Products, Inc), and 16 μg/mL of PHA-M. The feeding medium for the LDA was composed of 50% FCS, 50% Hu TCGD, 500 U/mL rhuIL-2, and 80 μg/ml PHA-M in RPMI 1640.

Ethyleneimine Oligomer Treatment of Whole Blood

Neat ethyleneimine oligomer was diluted aseptically with phosphate buffer to a 2% neutral solution. 2% solution of ethylene oligomer was used as a 20×working solution. 20×working solution was added to whole blood to yield a final concentration of 0.1% ethyleneimine oligomer. Other more dilute concentrations of ethyleneimine oligomer were obtained by diluting the 20×ethyleneimine oligomer stock in phosphate buffer prior to addition to the blood. The samples were rocked at room temperature for the indicated times. Equal volumes of phosphate buffer were added to control samples.

Enumeration and Analysis of PBMCs

The number of PBMCs was determined using an automated Coulter blood cell counter. Differentials were done on standard Wright-Giemsa stained smears.

Isolation of PBMC

After treatment with control buffer or ethyleneimine oligomer, the blood was diluted with an equal volume of Dulbecco's phosphate buffered saline (DPBS w/o Ca or Mg). The diluted blood was transferred to 50 mL conical tubes, and underlaid with 10 mL of Ficoll-Hypaque (Histopaque #1077, Sigma Chemical Co.). After centrifuging at 400×g for 30 minutes, the mononuclear band of cells at the interface of the histopaque/plasma boundary was harvested, transferred to sterile 50 mL conical tubes, and washed two times with 20 mL of DPBS at 400×g for 10 minutes. The cells were counted and adjusted to 1×10⁶ cells/mL in R20 medium and used for functional assays.

PHA Blastogenesis

PBMCs were assessed for their ability to proliferate after stimulation with PHA-M. Stock PHA-M was diluted to 1:40 in R20 medium. Cells and PHA-M were mixed in equal volumes (100 μL) in triplicate wells of a 96-well flat-bottom plate. After three days of culture in a humidified 10% CO₂ incubator, proliferation was evaluated by a 4 hour pulse with 1 μCi/well of [³H]-thymidine. The cells were harvested onto glass fiber filters with a multiple automated cell harvester. The filters were allowed to dry, and the level of thymidine incorporated was assessed by liquid scintillation counting.

Mixed Lymphocyte Cultures

Equal numbers of responder cells (100 μL at 1×10⁶ cells/mL) and stimulator cells were added per well in a flat bottom 96-well plate to establish the MLC. Stimulator cells had been prepared by treating 3 to 6×10⁶ PBMCs with 33 μg/mL of mitomycin C in a total volume of 3 mL R20 medium for 30 minutes at 37° C. The cells were then washed three times with 10 mL of R20 medium and resuspended at 1×10⁶ cells/mL. Control wells contained responder or stimulator cells alone with an additional 100 μL of R20 medium. Each sample was tested in triplicate. After five days of culture, the level of cell proliferation was assessed by [³H]-thymidine incorporation as described above for PHA blastogenesis.

Limiting Dilution Analysis

LDA using control and ethyleneimine oligomer-treated PBMCs was performed as follows. A γ-irradiated pool of allogeneic PBMCs was used as stimulator cells. This pool was prepared by irradiating PBMCs (obtained from 20 mL of blood from ten different donors) with 3000 rads using a cesium source. The irradiated cells were washed once with 10 mL of R20, and then pooled. The pooled cells were adjusted to 3×10⁷ cells/mL with ice cold R20. An equal volume of ice cold R20 containing 20% DMSO was added to the cells, and 1 mL aliquots were frozen. When needed the vials were thawed rapidly at 37° C., transferred to a 50 mL conical tubes on ice, and slowly diluted with 10 mL of R20 medium. The cells were counted, centrifuged at 300×g for 10 minutes and resuspended at 1×10⁶ cells/mL in 2×T-cell medium, and 1×10⁵ cells in 100 μL of medium were plated to each well of a 96-well flat bottom plate.

Limiting dilutions of the control- or ethyleneimine oligomer-treated responder cells were prepared as follows. Control cells were counted and adjusted to 10⁶ cells/mL. The cells were diluted 1:33 in R20 medium to bring the cells to 3×10⁴/mL, from which serial dilutions (1:3) were made to 3×10² cells/mL. Ethyleneimine oligomer-treated cells were adjusted to 1×10⁶ cells/mL in R20 medium, from which serial dilutions (1:10) were made to 10⁴ cells/mL. Aliquots of 100 μL of each responder cell dilution were plated into wells already containing the stimulator cells. For control cultures, 100 μL R20 medium was added to wells containing stimulator cells. The plates were incubated for three weeks at 37° C. in a humidified 10% CO² incubator. On days 3, 7, and 14, the wells were fed with 25 μL of LDA feed medium prepared as described above. In some experiments, PHA-M was not included in the feed medium on days 7 and 14. LDA plates were counted for clones, defined as tight clusters of cells, using an inverted microscope. The allostimulator control wells were used as baseline to calculate the number of positive wells for each dilution of control- or ethyleneimine oligomer-treated PBMCs. Cell frequencies were calculated by Poisson distribution analysis.

Annexin V Staining

Staining for the presence of phosphotidylserine on the surface of cells using Annexin V was done according to the manufacturer's instructions (PharMingen, San Diego, Calif.). PBMC to be stained were placed in R20 medium at 10⁶ cells/mL and cultured at 37° C. At the appropriate time points, 0.1 mL cells were removed per sample to be tested and washed once with 1 mL PBS. The cells were resuspended in 100 μL PBS. 10 μL of resuspended cells were added to a tube containing 90 μL binding buffer, 2 μL Annexin V FITC and 5 μL propidium iodide. The cells were incubated for 15 minutes in the dark, 300 μL of binding buffer was added, and the cells then analyzed on the flow cytometer within one hour.

TUNEL Staining

Staining for the presence of DNA fragments was performed according to the instructions of the manufacturer (R & D Systems, Minneapolis, Minn.). Briefly, control- or ethyleneimine oligomer-treated PBMCs were tested directly by incubating 1×10⁶ cells with 1 mL 3.7% formaldehyde for 10 minutes at room temperature. The cells were pelleted and resuspended in 1 mL of PBS for a two minute incubation. The cells were then pelleted and resuspended in 100 μL CYTOPORE™ for 30 minutes at room temperature or stored overnight at 4° C. The positive control was prepared by washing one aliquot of cells with DNase-free water, then resuspending the cells in 25 μL of TACS-Nuclease working solution. This tube was incubated at 37° C. for 30 minutes. The cells were then washed in DNase-free water (300×g for 5 minutes), resuspended in 1 mL of labeling buffer, and washed again. The cells were then resuspended in 25 μL of labeling reaction mix. The tubes were incubated for one hour at 37° C., and then 1 mL of stop buffer was added. After centrifugation, the cells were resuspended in 25 μL of diluted strepavidin-FITC working solution and incubated in the dark for ten minutes at room temperature. The cells were then washed and resuspended in 500 μL of DPBS. The samples were stored on ice in the dark until flow cytometric analysis, which was done within two hours after the completion of the assay.

### EXAMPLE 5

### Ethyleneimine Oligomer Treatment of Murine Spleen Cells

Studies are performed to determine whether in vitro treatment of murine spleen cells with ethyleneimine oligomer (0.1% for 1 hour) results in the functional inactivation of these cells.

Treatment with ethyleneimine oligomer results in the failure of phorbol ester to activate cells and induce the upregulation of CD69 expression. Ethyleneimine oligomer treatment abrogates the proliferative response of splenocytes to mitogen ConA stimulation or allogeneic stimulator cells. In addition, ethyleneimine oligomer treated cells do not stimulate proliferation of allogeneic responder cells. These findings indicate that ethyleneimine oligomer functionally inactivates murine splenocytes as effectively as human PBMC.

### EXAMPLE 6

### Ethyleneimine Oligomer Treatment of Allogeneic Donor Cells on Donor and Recipient in vivo Immune Responses

The effect ethyleneimine oligomer treatment on the ability of spleen cells to elicit in vivo immune responses is investigated in a murine model of transfusion-associated graft-versus-host disease (TAGVHD). The model utilizes injection of parental splenocytes into F₁ hybrid recipients created from two genetically distinct parental strains.

The effect of ethyleneimine oligomer treatment is examined in B6D2F₁ hybrid mice. Parental C57BL/6 cells or DBA/2 cells were alternatively injected into the hybrids. Injection of C57BL/6 cells into G62 F₁ recipients results in an acute form of GVHD that is characterized by splenomegaly, generation of donor anti-recipient cytolytic T lymphocytes (CTL), and a reaction that resembles TAGVHD. Injection of DBA/2 cells into B6D2 F₁ hybrid mice results in a chronic form of GVHD that is characterized by splenomegaly, hyperproduction of immunoglobulin, and a condition that resembles lupus.

The effect of ethyleneimine oligomer treatment on the ability to induce GVHD-associated responsiveness is assessed by examining spleen mass, cytolytic activity, and immunoglobulin production in F₁ hybrid mice following injection of injection of parental splenocytes into F₁ hybrid recipients. The results are shown in Table 4:

TABLE 4

| | | | Cytolytic | Immunoglobulin | |
|---|---|---|---|---|---|
| | | Spleen mass | Activity (% | Production | Number of |
| Donor | Treatment | (mg) | lysis) | (treated/control) | Mice |
| C57BL/6 | Control | 0.163 ± 0.063 | 49.5 ± 21.8 | 1.18 ± 0.68 | 6 |
| C57BL/6 | Ethyleneimine | 0.065 ± 0.013 | 2.3 ± 3.2 | 0.83 ± 0.37 | 9 |
| | oligomer | | | | |
| DBA/2 | Control | 0.131 ± 0.020 | −0.9 ± 1.8 | 21.5 ± 8.9 | 7 |
| DBA/2 | Ethyleneimine | 0.064 ± 0.01 | −1.1 ± 2.2 | 1.04 ± .034 | 9 |
| | oligomer | | | | |
| None | Control | 0.065 ± 0.08 | 1.88 ± 2.8 | 0.99 ± 0.27 | 9 |

These results demonstrate that ethyleneimine oligomer treatment results in inhibition of splenomegaly and inhibition of CTL activity when C57BL/6 donor cells are used. Ethyleneimine oligomer treatment of DBA/2 cells results in inhibition of splenomegaly and a decrease in generalized immunoglobulin production as compared to untreated controls.

The effect of ethyleneimine oligomer treatment on the ability to induce IgG alloantibody responses to donor antigens is also examined using C57BL/6 donor cells and DBA/2 recipients. The results from three studies are presented in Table 5:

TABLE 5

IgG Alloantibody Levels

| Trial | Control | Ethyleneimine Oligomer |
|---|---|---|
| 1 | 130 ± 49 | −7 ± 6 |
| 2 | 128 ± 22 | −8 ± 3 |
| 3 | 203 ± 70 | 4 ± 7 |

Treatment of donor cells with ethyleneimine oligomer results in significantly lower levels of IgG alloantibodies.

These results demonstrate that ethyleneimine oligomer treatment of allogeneic donor lymphoid cells interferes with both the in vivo ability of the donor cells to respond and the in vivo ability of the donor cells to induce recipient immune responses.

Other embodiments are within the claims.

What is claimed is:

1. A method for treating a patient having an immune dysfunction, said method comprising the steps of:
(a) treating peripheral blood mononuclear cells with an effective amount of an aziridino-containing compound; and
(b) administering said peripheral blood mononuclear cells to said patient.

2. The method of claim 1, wherein said immune dysfunction is cutaneous T-cell lymphoma, graft versus host disease, allograft rejection following organ transplantation, systemic lupus erythematosus, systemic sclerosis, inflammatory bowel disease, or rheumatoid arthritis.

3. The method of claim 1 wherein said compound has the formula (II): 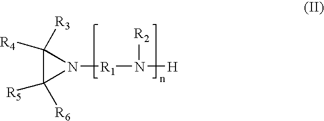
wherein each R₁ is a divalent hydrocarbon moiety containing between 2 and 4 carbon atoms, inclusive; each of R₂, R₃, R₄, R₅, and R₆ is, independently, H or a monovalent hydrocarbon moiety containing between 1 arid 4 carbon atoms; and n is an integer between 1 and 10, inclusive.

4. The method of claim 1, wherein said compound is ethyleneimine dimer.

5. The method of claim 1, wherein said compound is an ethyleneimine trimer.

6. The method of claim 1, wherein said compound is an ethyleneimine tetramer.

7. The method of claim 1, wherein said compound has the formula (III): 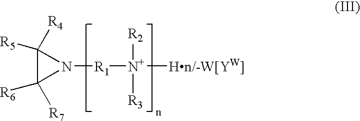
wherein each R₁ is a divalent hydrocarbon moiety containing between 2 and 4 carbon atoms, inclusive; each of R₂, R₃, R₄, R₅, B₆, and R₇ is, independently, H or a monovalent hydrocarbon moiety containing between 1 and 4 carbon atoms; X is Cl or Br, Y is a pharmaceutically acceptable counter anion; W is valency of Y; and n is an integer between 1 and 10, inclusive.

8. The method of claim 1, wherein said compound has the formula (IV); 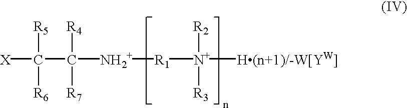
wherein each R1 is a divalent hydrocarbon moiety containing between 2 and 4 carbon atoms, inclusive; each of R₂, R₃, R₄, R₅, B₆, and R₇ is, independently, H or a monovalent hydrocarbon moiety containing between 1 and 4 carbon atoms; X is Cal or Br; Y is a pharmaceutically acceptable counter anion; W is valency of Y; and n is an integer between 1 and 10, inclusive.

9. A method for treating a patient having an immune dysfunction, said method comprising the steps of:
(a) extracorporeally treating peripheral blood mononuclear cells from said patient with an effective amount of an aziridino-containing compound;
(b) separately said peripheral blood mononuclear cells from said aziridino-containing compound; and
(c) administering said peripheral blood mononuclear cells to said patient.

10. A method for preventing graft-versus-host (GVH) disease in a patient, the method comprising the steps of:
(a) extracorporeally treating a blood composition with an effective amount of an aziridino-containing compound; and
(b) administering said treated blood cell population to said patient,
thereby preventing GVH disease in said patient.

11. The method of claim 10, wherein said blood composition comprises peripheral blood mononuclear cells (PBMC).

12. The method of claim 10, wherein said blood composition is a non-leukoreduced blood cell concentrate.

13. The method of claim 10, wherein said blood composition is a heterologous blood cell population.

14. The method of claim 10, wherein said method further separating said aziridino-containing compound from said treated blood cell composition prior to administering said treated blood composition to said patient.

15. The method of claim 14, wherein at least 99% of said aziridino-containing compound is removed from said treated blood cell composition prior to administering said treated blood composition to said patient.

16. The method of claim 10, wherein said compound has the formula (II): 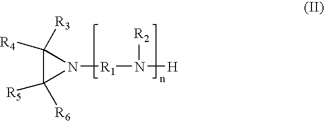
wherein each R₁ is a divalent hydrocarbon moiety containing between 2 and 4 carbon atoms, inclusive; each of R₂, R₃, R₄, R₅, and R₆ is, independently, H or a monovalent hydrocarbon moiety containing between 1 arid 4 carbon atoms; and n is an integer between 1 and 10, inclusive.

17. The method of claim 10, wherein said compound is an ethyleneimine dimer.

18. The method of claim 10, wherein said compound is an ethyleneimine trimer.

19. The method of claim 10, wherein said compound is an ethyleneimine tetramer.

20. The method of claim 10, wherein said compound has the formula (III): 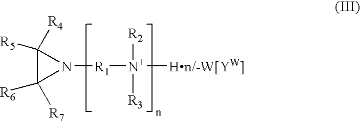
wherein each R₁ is a divalent hydrocarbon moiety containing between 2 and 4 carbon atoms, inclusive; each of R₂, R₃, R₄, R₅, B₆, and R₇ is, independently, H or a monovalent hydrocarbon moiety containing between 1 and 4 carbon atoms; X is Cl or Br, Y is a pharmaceutically acceptable counter anion; W is valency of Y; and n is an integer between 1 and 10, inclusive.

21. The method of claim 10, wherein said compound has the formula (IV); 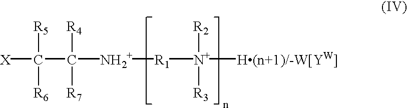
wherein each R1 is a divalent hydrocarbon moiety containing between 2 and 4 carbon atoms, inclusive; each of R₂, R₃, R₄, R₅, B₆, and R₇ is, independently, H or a monovalent hydrocarbon moiety containing between 1 and 4 carbon atoms; X is Cal or Br; Y is a pharmaceutically acceptable counter anion; W is valency of Y; and n is an integer between 1 and 10, inclusive.

22. The method of claim 10, wherein said patient is a human.

23. The method of claim 10, wherein said patient suffers from or is at risk for immune dysfunction.

24. The method of claim 22, wherein said human patient suffers from or is at risk for immune dysfunction.

25. A method for preventing graft-versus-host (GVH) disease in a patient, the method comprising the steps of:
(a) treating a heterologous blood composition with an effective amount of an ethylene oligomer compound;
(b) removing said ethylene oligomer from said heterologous treated blood composition; and
(c) administering said treated blood cell population to said patient,
thereby preventing GVH disease in said patient.

26. The method of claim 25, wherein said patient is a human.

27. The method of claim 25, wherein said compound is an ethyleneimine dimer.

28. The method of claim 25, wherein said compound is an ethyleneimine trimer.

29. The method of claim 25, wherein said compound is an ethyleneimine tetramer.

30. A method for treating graft-versus-host (GVH) disease in a patient, the method comprising the steps of:
(a) treating a heterologous blood composition with an effective amount of an aziridino-containing compound; and
(b) administering said treated blood cell population to said patient,
thereby treating GVH disease in said patient.

31. A method for preventing graft-versus-host (GVH) disease in a patient, the method comprising the steps of:
(a) treating a heterologous blood composition with an effective amount of an ethylene oligomer compound;
(b) removing said ethylene oligomer from said heterologous treated blood composition; and
(c) administering said treated blood cell population to said patient,
thereby preventing GVH disease in said patient.

32. A method for preventing an alloantibody response in a patient, the method comprising the steps of:
(a) treating a heterologous blood composition with an effective amount of an aziridino-containing compound; and
(b) administering said treated blood cell population to said patient,
thereby preventing said alloantibody response in said patient.

33. A method for functionally inactivating a leukocyte in a patient, the method comprising the steps of:
(a) treating a heterologous blood composition comprising a leukocyte with an effective amount of an aziridino-containing compound; and
(b) administering said treated blood cell population to said patient,
thereby inactivating said leukocyte in said patient.

34. The method of claim 33, wherein said leukocyte renders does not proliferate following said treatment.

35. A blood composition produced by treating a composition comprising peripheral blood mononuclear cells with a non-viricidal amount of an aziridino-containing compound. wherein said aziridino-containing compound is present in an amount sufficient to inhibit proliferation of said peripheral blood mononuclear cells.
